(19) United States
(12) Patent Application Publication
(10) Pub. No.: US 2024/0090893 A1
(43) Pub. Date: Mar. 21, 2024

(54) Suture Ring and Finger Protector

(71) Applicant: Arthrex, Inc., Naples, FL (US)

(72) Inventors: Tal S. DAVID, San Diego, CA (US); Peter J. DREYFUSS, Naples, FL (US); John SEYLER, Naples, FL (US)

(73) Assignee: Arthrex, Inc., Naples, FL (US)

(21) Appl. No.: 17/932,448

(22) Filed: Sep. 15, 2022

(51) Int. Cl. A61B 17/04

(52) U.S. Cl. CPC A61B 17/0483; A61B 17/0401; A61B 17/0466; A61B 17/0493; A61B 2017/0023; A61B 2017/0496

(57) ABSTRACT

A surgical construct provides a dual purpose of a suture retainer and a finger protector. The surgical construct efficiently allows for surgeon's protection when using tensionable fixation devices (such as tensionable anchors) without extra steps or added cost. The surgical construct can retain suture and can be also worn on fingers to prevent glove and/or finger cuts when tensioning sutures.

### FIELD OF THE INVENTION

The disclosure relates to the field of surgery and, more specifically, to surgical constructs and methods of surgery.

### SUMMARY

Surgical constructs and methods for surgical repairs are disclosed. A surgical construct provides a dual purpose of a suture ring and a finger protector. A surgical construct allows a surgeon efficient protection when using tensionable fixation devices.

Methods of suture tensioning are also disclosed. A surgical construct provides suture retention as well as finger protection while conducting suture tensioning.

### BRIEF DESCRIPTION OF THE DRAWINGS

FIGS. 1 and 2 illustrate views of a surgical assembly according to an exemplary embodiment.

FIG. 3 illustrates a surgical construct according to an exemplary embodiment.

FIGS. 4 and 5 illustrate views of the surgical construct of FIG. 3 positioned on a finger.

FIG. 6 is an enlarged view of the inserter handle of the surgical assembly of FIG. 1.

FIG. 7 illustrates a surgical assembly according to another exemplary embodiment.

FIG. 8 illustrates a surgical construct according to another exemplary embodiment.

FIG. 9 is an enlarged view of an inserter handle of a surgical assembly with the surgical construct of FIG. 8.

### DETAILED DESCRIPTION

The disclosure provides surgical assemblies, constructs, and methods of tissue repairs. A surgical construct provides a dual purpose of a suture retainer and a finger protector. A surgical construct efficiently allows for surgeon's protection when using tensionable fixation devices (such as tensionable anchors) without extra steps or added cost. A surgical construct can retain suture and can be also worn on fingers to prevent glove and/or finger cuts when tensioning sutures.

Methods of manipulating flexible strands during tissue repairs are also disclosed. An exemplary method comprises inter alia: (i) retaining, with a surgical construct, a flexible strand on an inserter handle; and (ii) placing the surgical construct on a finger to prevent glove and/or finger cuts and/or skin injuries when tensioning the flexible strand. The method can further include attaching the surgical construct to the inserter handle to securely engage and retain the flexible strand; removing the surgical construct from the inserter handle; inserting the finger through apertures of the surgical construct so that the surgical construct covers at least a portion of a finger surface; and tensioning the flexible strand. The method can further include the step of removing the surgical construct from the finger after the step of tensioning the flexible strand.

The surgical construct is configured to provide both the function of a suture ring (suture retainer) and of a finger protector (finger guard or shield). A surgical construct can have a plurality of apertures with an O-ring configuration. The O-ring can be attached on the back of an inserter handle for inserting a fixation device into tissue such that, after insertion of the fixation device and O-ring removal, the surgical construct can be worn on fingers to prevent glove/finger cuts when tensioning sutures. A surgical construct can be a dual-purpose suture ring and finger protector. A surgical construct can be a dual-purpose handle suture retaining ring and finger protector. A surgical construct can be reusable. A surgical construct can be a single-use construct. A surgical construct can be a one-piece flexible construct.

The flexible strand can be any flexible coupler attached to a fixation device, for example, suture attached to a knotless, tensionable fixation device. The fixation device can be any knotless suture anchor such as, for example, the two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of all of which are fully incorporated by reference in their entirety herein. A knotless fixation device comprises an anchor body (or screw) and an eyelet.

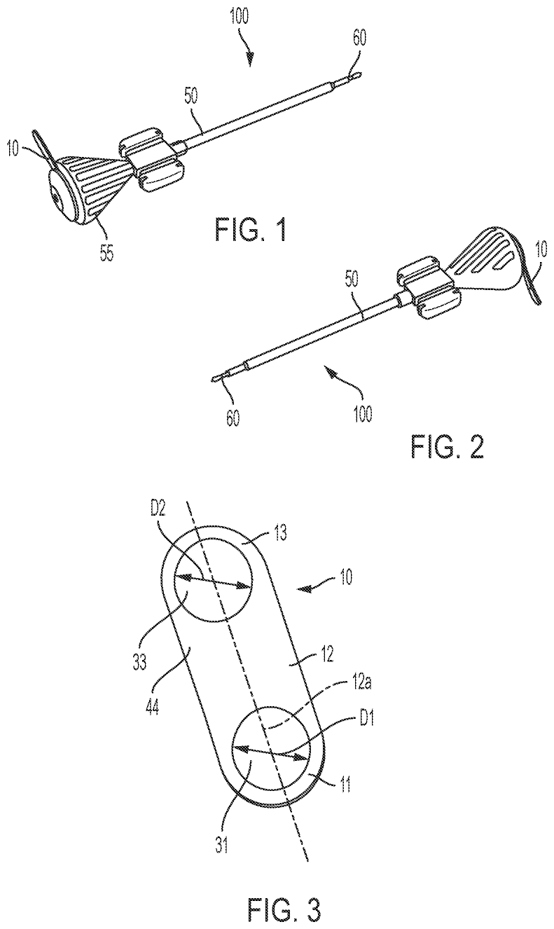

Referring now to the drawings, where like elements are designated by like reference numerals, FIGS. 1-9 illustrate surgical construct 10, 110 employed with surgical assembly 100, 200 (FIGS. 1, 2 and 7) of the present disclosure.

FIGS. 1 and 2 illustrate exemplary surgical assembly 100 which includes surgical inserter 50 with an attached fixation device 60. Fixation device 60 can be a suture anchor, for example, a knotless suture anchor for insertion into tissue such as bone. In exemplary embodiments, fixation device 60 is a knotless, tensionable suture anchor such as the two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of all of which are fully incorporated by reference in their entirety herein. A knotless fixation device comprises an anchor body (or screw) and an eyelet that allows passage of at least one flexible strand (flexible coupler) 20, as detailed below. Surgical construct 10 is securely attached to handle 55 of surgical inserter 50, to engage, retain, and secure at least one flexible strand 20.

Reference is now made to FIG. 3 which is a top view of exemplary surgical construct 10 (suture retainer 10; suture ring 10; O-ring 10; finger protector 10; finger shield 10; finger guard 10; suture ring/finger protector 10) of the present disclosure.

Surgical construct 10 comprises an elongated oval member 12 or body 12 having a longitudinal axis 12a, a proximal end 11, a distal end 13. Body 12 may be a one-piece construct manufactured from a flexible material (e.g., silicone) which enables the construct to be deformed and/or otherwise easily attached to and detached from an insertion tool and also secured on a finger. Body 12 can be flexible or partially flexible. Body 12 can be constructed of a flexible, resilient material that is impervious to liquids and bodily fluids and is also adaptable to a range of finger diameters. Body 12 can be constructed to conform to the ventral surface of a finger when worn on the finger.

As depicted in FIG. 3, body 12 is provided with a plurality of first and second apertures, openings, through-holes 31, 33 located about symmetrically relative to the longitudinal axis 12a and at the proximal end 11 and distal end 13, respectively. First and second through-holes 31, 33 extend from a top surface to a bottom surface of body 12. In an embodiment, first and second apertures 31, 33 have a generally round configuration. As depicted in FIG. 3, diameter D1 of first aperture 31 is larger than diameter D2 of second aperture 33, to allow easier insertion and accommodation of the anatomy of a finger when surgical construct 10 acts as a finger protector. Each of the first and second apertures 31, 33 acts as an O-ring for suture retention when surgical construct 10 engages handle 55 of insertion tool 50.

Body 12 also includes a finger guard 44 or finger padding/cushion 44 extending between the first and second apertures 31, 33. The finger guard 44 allows a surgeon efficient protection when surgical construct 10 is worn on a finger during tensioning of tensionable suture anchors.

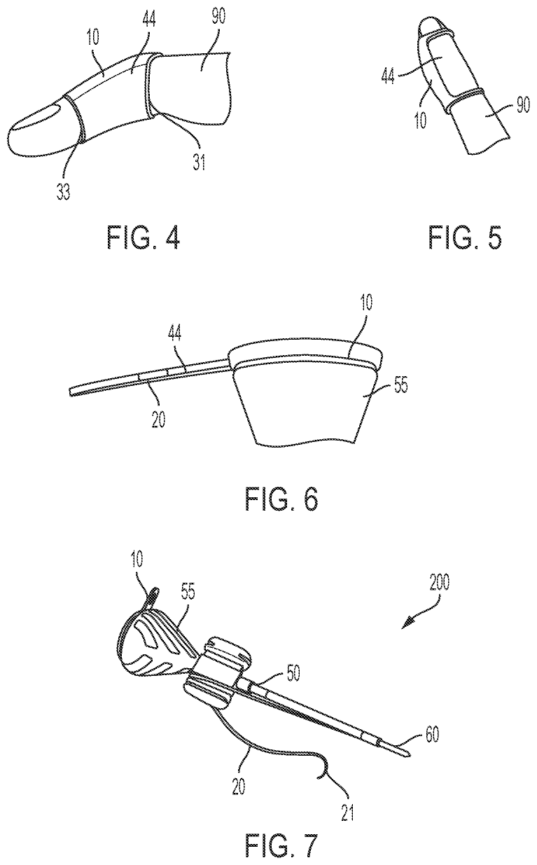

FIGS. 4 and 5 schematically illustrate surgical construct 10 positioned on a surgeon's finger 90 to prevent glove and/or finger cuts when the surgeon tensions flexible strands (sutures). Surgical construct 10 can be positioned over a glove covering the hand or directly on the finger 90, as desired. Surgeon's finger is inserted into first aperture 31, followed by second aperture 33, to allow finger guard 44 to rest on a ventral surface of the finger.

FIG. 6 illustrates surgical construct 10 secured to handle 55 of insertion tool 50, to allow insertion of fixation device 60 (suture anchor 60) with flexible strand 20 into bone and the subsequent tensioning of the flexible strand 20. Thus, surgical construct 10 is configured to provide both the function of a suture ring (suture retainer) as shown in FIG. 6, for example, and of a finger protector (finger guard or shield) as shown in FIG. 4, for example. After insertion of the fixation device 60 and O-ring removal, surgical construct 10 can be worn on fingers 90 to prevent glove/finger cuts when tensioning flexible strands. Surgical construct 10 can be a dual-purpose suture ring and finger protector. Surgical construct 10 can be reusable or disposable.

FIG. 7 illustrates assembly 200 which is about similar to assembly 100 detailed above in that it also includes surgical construct 10 positioned onto handle 55 of insertion tool 50; however, assembly 200 also includes surgical needle 21 attached to flexible strand 20.

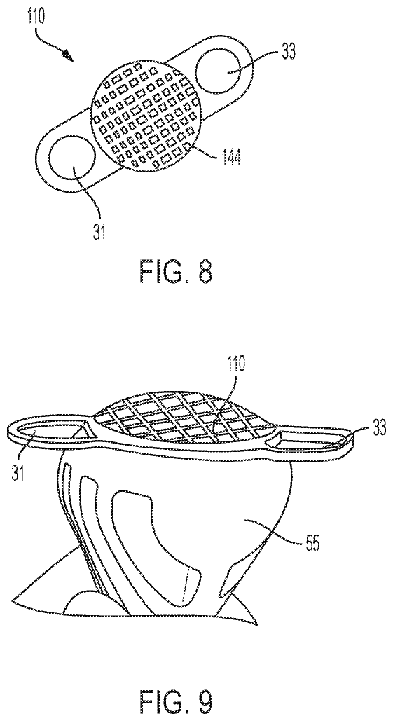

FIGS. 8 and 9 illustrate yet another surgical construct 110 according to another exemplary embodiment. Surgical construct 110 (suture retainer 110; suture ring 110; O-ring 110; finger protector 110; finger shield 110; finger guard 110; suture ring/finger protector 110) is about similar to surgical construct 10 detailed above in that surgical construct 110 also has a dual function of suture retainer and finger protector, and with a configuration about similar to that of surgical construct 10. However, surgical construct 110 is provided with finger guard 144 or finger padding/cushion 144 extending between the first and second apertures 31, 33 that has a round configuration designed to fit and cover a most proximal end of handle 55 (as shown in FIG. 9). The finger guard 144 acts as an O-ring (retaining suture) and also allows a surgeon efficient protection when surgical construct 110 is worn on a finger during tensioning of tensionable suture anchors.

As in the previously described embodiment, body 12 of construct 110 can be a one-piece construct manufactured from a flexible material (e.g., silicone) which enables the construct to be deformed and/or otherwise easily attached to and detached from an insertion tool and also secured on a finger. The body can be flexible or partially flexible, and can be manufactured from a flexible, resilient material that is impervious to liquids and bodily fluids and is also adaptable to a range of finger diameters. Finger guard 144 can be formed of a material similar to the material defining first and second through-holes 31, 33. Finger guard 144 can be formed of a material different from the material defining first and second through-holes 31, 33. If desired, finger guard 144 can include a plurality of perforations or apertures to allow increased deformation when secured to an inserter handle and a better grip for the surgeon. Finger guard 144 can consist essentially of silicone. Finger guard 144 can consist essentially of fabric or textile material.

An exemplary method of tensioning suture comprises inter alia: (i) retaining, with an O-ring 31, 33 of surgical construct 10 and/or a finger guard 144 of surgical construct 110, a flexible strand 20 on an inserter handle 55; and (ii) placing the surgical construct 10, 110 on a finger 90 to prevent glove and/or finger cuts and/or skin injuries when tensioning the flexible strand 20. The method can further include attaching the surgical construct 10 to the inserter handle 55 to securely engage and retain the flexible strand 20 attached to a tensionable fixation device 60 by one of the O-rings 31, 33 and/or by the finger guard 144; securing the tensionable fixation device 60 into bone; removing the surgical construct 10 from the inserter handle 55; inserting finger 90 through apertures 31, 33 of surgical construct 10, 110 so that finger padding 44, 144 rests and extends over at least a portion of a surface of finger 90; and tensioning the flexible strand 20. The method can further include the step of removing the surgical construct 10, 110 from the finger 90 after the step of tensioning the flexible strand 20.

Flexible strand 20 can include a single filament, or fiber, or can include multiple continuous filaments, segments or regions of filaments that have different configurations (for example, different diameters and/or different compositions). The filament regions/segments may each be homogenous (i.e., formed of a same material) or may be a combination of homogenous and heterogenous (i.e., formed of a plurality of materials).

Flexible strand 20 can be made of any known suture construct, such as multifilament, braided, knitted, woven suture, or including fibers of ultrahigh molecular weight polyethylene (UHMWPE) or the FiberWire® suture (disclosed in U.S. Pat. No. 6,716,234, the disclosure of which is hereby incorporated by reference in its entirety herein). The flexible strand may be also formed of suture tape, for example, Arthrex FiberTape®, which is a high strength suture tape that is braided and rectangular-like in cross section and as disclosed in U.S. Pat. No. 7,892,256, the disclosure of which is incorporated by reference in its entirety herein.

Flexible strand 20 can also include, and be manufactured with, any kind of material (suture, nylon, silk, UHMWPE, metal, bioabsorbable, etc.) that can allow the flexible strand to be secured to an inserter handle by surgical construct 10.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical construct for suture retention and finger protection, comprising:
a flexible body with an elongated configuration, a longitudinal axis, a proximal end, and a distal end; and
a first aperture located at the proximal end, and a second aperture located at the distal end.

2. The surgical construct of claim 1, wherein at least one of the first aperture and the second aperture is an O-ring for attachment to a handle of an insertion tool.

3. The surgical construct of claim 1, wherein the first aperture has a diameter larger than the diameter of the second aperture, to accommodate a finger.

4. The surgical construct of claim 1, wherein the flexible body consists essentially of silicon.

5. The surgical construct of claim 1, wherein the surgical construct is disposable.

6. The surgical construct of claim 1, wherein at least one of the first aperture and the second aperture is configured to engage a handle of an insertion tool, to retain and secure a flexible strand on the insertion tool.

7. The surgical construct of claim 6, wherein the insertion tool is an inserter for a tensionable suture anchor.

8. The surgical construct of claim 6, wherein the flexible strand is high-strength suture.

9. The surgical construct of claim 1, wherein at least a portion of the flexible body extending between the first aperture and the second aperture is configured to engage a handle of an insertion tool, to retain and secure a flexible strand on the insertion tool.

10. The surgical construct of claim 9, wherein the insertion tool is an inserter for a tensionable suture anchor.

11. The surgical construct of claim 9, wherein the flexible strand is high-strength suture.

12. A method of tensioning suture, comprising:
securing a surgical construct on a handle of an inserter, the surgical construct including a flexible body and first and second through-holes extending from a top surface to a bottom surface of the flexible body;
advancing the inserter in the vicinity of a surgical site;
securing a fixation device attached to the inserter into tissue at the surgical site while a flexible strand of the fixation device is retained on the handle of the inserter by the surgical construct;
removing the surgical construct from the handle of the inserter; and
removably attaching the surgical construct to a finger or thumb, to protect the finger or the thumb during tensioning of the flexible strand.

13. The method of claim 12, wherein the surgical construct is a one-piece construct.

14. The method of claim 12, wherein the surgical construct is a suture retainer and finger protector.

15. The method of claim 12 further comprising deforming the surgical construct to removably attach and detach one of the first and second through-holes to the handle.

16. The method of claim 12 further comprising deforming a region of the surgical construct located between the first and second through-holes, to removably attach and detach the region to the handle.

17. The method of claim 12 further comprising inserting the finger or thumb through each of the first and second through-holes.

18. The method of claim 12, wherein the flexible strand is suture or suture tape.
